(19) United States
(12) Patent Application Publication
(10) Pub. No.: US 2026/0182906 A1
(43) Pub. Date: Jul. 2, 2026

(54) ELECTRONIC DEVICE FOR DETERMINING SKIN TONE THROUGH PHOTOPLETHYSMOGRAM

(71) Applicant: Samsung Electronics Co., Ltd., Suwon-si (KR)

(72) Inventors: Jongboo KIM, Suwon-si (KR); Raeyoung KIM, Suwon-si (KR); Jeongho PARK, Suwon-si (KR); Radwanul Hasan SIDDIQUE, San Jose, CA (US); Shailabh KUMAR, San Jose, CA (US); Wonhyuk JUNG, Suwon-si (KR)

(73) Assignee: Samsung Electronics Co., Ltd., Suwon-si (KR)

(21) Appl. No.: 19/003,299

(22) Filed: Dec. 27, 2024

(51) Int. Cl. A61B 5/00

(52) U.S. Cl. CPC A61B 5/443

(57) ABSTRACT

An electronic device includes a light-emitting module including a first polarizer, the light-emitting module configured to emit first light polarized in the first direction to a skin of the user, through the first polarizer, a first light-receiving module including a second polarizer, the first light-receiving module configured to receive second light which is the first light reflected from the user, through the second polarizer, a second light-receiving module configured to receive third light which is the first light reflected from the user, a sensing circuit configured to generate a first PPG signal based on the second light received by the first light-receiving module and generate a second PPG signal based on third light received by the second light-receiving module, and a processor configured to distinguish a skin tone of the user based on a difference between the first PPG signal and the second PPG signal.

### BACKGROUND

The inventive concepts relate to an electronic device to distinguish a skin tone of a human body by using a photoplethysmogram (PPG).

Atrial fibrillation (AF), which is an intra-atrial disease which causes rapid and irregular heartbeats, may result in serious physical dangers such as thrombi and/or cerebrovascular accidents. To detect irregular cardiac rhythms such as atrial fibrillation or atrial flutter (AFL), detection algorithms based on various biometric signals (e.g., an electrocardiogram (ECG) and a photoplethysmogram (PPG)) have been developed.

A PPG is an algorithm for detecting heartbeats and/or the like based on intensity of reflected light emitted from an electronic device (e.g., a wearable device). As sensitivity to wave lengths of light (e.g., ultraviolet light) varies according to a skin tone, the degree of transmission of light through the skin varies according to a skin tone. Accordingly, it is beneficial to distinguish a skin tone of a user for improvement in the accuracy of PPG or the calculation of an amount of ultraviolet absorption by compensating for said variations.

### SUMMARY

The inventive concepts provide an electronic device to distinguish a skin tone of a user based on a degree of change in photoplethysmogram (PPG) performance according to skin tones.

The technical goals are not limited to the aforementioned technical goals, and other technical goals not mentioned above may be clearly understood to those skilled in the art based on the following descriptions.

According to an aspect of the inventive concepts, there is provided an electronic device including a light-emitter comprising a first polarizer polarized in a first direction, the light-emitter configured to emit first light polarized in the first direction through the first polarizer to a skin of a user; a first light-receiver comprising a second polarizer polarized in a second direction different from the first direction, the first light-receiver configured to receive second light reflected from the skin of the user through the second polarizer; a second light-receiver configured to receive third light reflected from the skin of the user; and processing circuitry configured to generate a first photoplethysmogram (PPG) signal based on the second light received by the first light-receiver, to generate a second PPG signal based on the third light received by the second light-receiver, and to distinguish a skin tone of the user based on a difference between the first PPG signal and the second PPG signal.

According to another aspect of the inventive concepts, there is provided an electronic device including a first light-emitter comprising a first polarizer polarized in a first direction, the light-emitter configured to emit first light polarized in the first direction through the first polarizer to a skin of a user; a second light-emitter configured to emit second light to the skin of the user; a light-receiver comprising a second polarizer polarized in a second direction different from the first direction, the light-receiver configured to receive third light, which is the first light reflected from the user, through the second polarizer, and to receive fourth light through the second polarizer, the third light including the first light reflected from the skin of the user and the fourth light including the second light reflected from the skin of the user; processing circuitry configured to generate a first photoplethysmogram (PPG) signal based on the third light received by the light-receiver, to generate a second PPG signal based on the fourth light received by the light-receiver, and to distinguish a skin tone of the user based on a difference between the first PPG signal and the second PPG signal.

According to another aspect of the inventive concepts, there is provided an electronic device including a light-emitter configured to emit first light to a skin of a user based on a first driving current during a first period, and to emit second light to the skin of a user based on a second driving current during a second period different from the first period; a light-receiver configured to receive third light in the first period and to receive fourth light in the second period, the third light including the first light reflected from the skin of the user, and the fourth light including the second light reflected from the skin of the user; and processing circuitry configured to output the first driving current in the first period, to output the second driving current in the second period, to generate a first photoplethysmogram (PPG) signal based on the third light received by the light-receiver in the first period, to generate a second PPG signal based on the fourth light received by the light-receiver in the second period, and to distinguish a skin tone of the user based on a difference between the first PPG signal and the second PPG signal, wherein the second driving current has an intensity different from an intensity of the first driving current.

### BRIEF DESCRIPTION OF THE DRAWINGS

Embodiments will be more clearly understood from the following detailed description taken in conjunction with the accompanying drawings in which:

FIG. 1 is a block diagram illustrating an electronic device according to at least one embodiment;

FIG. 2 is a graph showing a photoplethysmogram (PPG) signal according to at least one embodiment;

FIGS. 3A and 3B are graphs showing changes in PPG performance according to at least one embodiment;

FIGS. 4A and 4B are graphs showing changes in PPG performance according to at least one embodiment;

FIGS. 5A and 5B are block diagrams each illustrating a PPG sensor according to at least one embodiment;

FIG. 6 is a flowchart for describing an operating method of an electronic device, according to at least one embodiment;

FIGS. 7A and 7B are block diagrams each illustrating a PPG sensor according to at least one embodiment;

FIG. 8 is a flowchart for describing an operating method of an electronic device, according to at least one embodiment;

FIGS. 9A and 9B are block diagrams each illustrating a PPG sensor according to at least one embodiment;

FIG. 10 is a graph showing changes in PPG performance according to at least one embodiment; and

FIG. 11 is a flowchart for describing an operating method of an electronic device, according to at least one embodiment.

### DETAILED DESCRIPTION OF THE EMBODIMENTS

Hereinafter, one or more embodiments will be described in detail with reference to the accompanying drawings. Like reference numerals in the drawings denote like components, and therefore repeat descriptions thereof will be omitted. Some sizes of components in the drawings may be exaggerated for convenience of explanation. In addition, embodiments to be described below are only examples, and various modifications from such embodiments may be possible. Additionally, although the terms “first,” “second,” “third,” etc., may be used herein to describe various elements, components, regions, layers, and/or sections, these elements, components, regions, layers, and/or sections, should not be limited by these terms as these terms are only used to distinguish one element, component, region, layer, or section, from another region, layer, or section. Thus, a first element, component, region, layer, or section, discussed below may be termed a second element, component, region, layer, or section, without departing from the scope of this disclosure.

Additionally, functional elements, including those modified by the use of “unit”, “circuit”, “module”, and/or the like, that enable the functions described below may be implemented and/or supported by processing circuitry such as, hardware, software, or a combination of hardware and software. For example, the processing circuitry may include, but is not limited to, a central processing unit (CPU), an application processor (AP), an arithmetic logic unit (ALU), a graphic processing unit (GPU), a digital signal processor, a microcomputer, a field programmable gate array (FPGA), a System-on-Chip (SoC) a programmable logic unit, a microprocessor, or an application-specific integrated circuit (ASIC), active elements (e.g., transistors, gates, etc.), passive elements (e.g., capacitors, inductors, etc.), and/or the like.

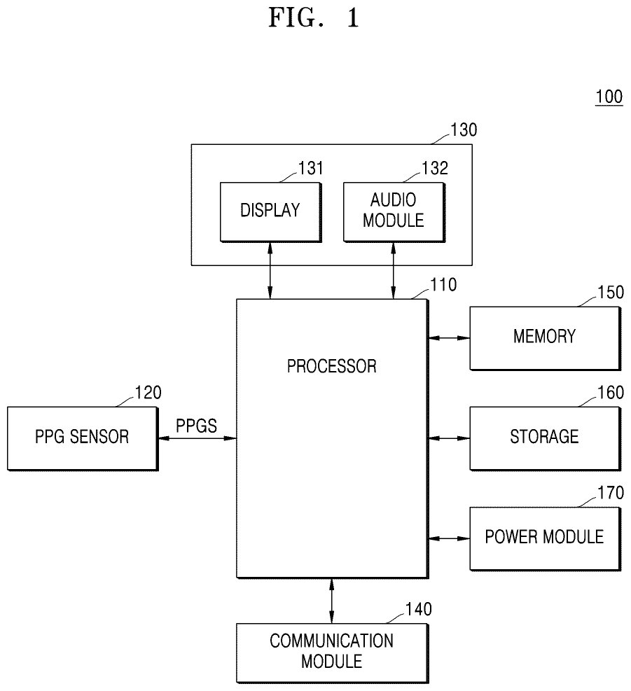

FIG. 1 is a block diagram illustrating an electronic device 100 according to at least one embodiment.

Referring to FIG. 1, the electronic device 100 may include a processor 110, a photoplethysmogram (PPG) sensor 120, an input/output device 130, a communication module 140, a memory 150, a storage 160, and a power module 170. However, the electronic device 100 is not limited thereto and may further include various components.

The electronic device 100 may include a user-wearable device configured to monitor biometric signals of the user. The user may wear the electronic device on a body part such as an arm, a leg, a neck and/or the like; and the electronic device 100 may be configured to sense the physiological signals of the user and monitor health conditions of the user through the PPG sensor 120. However, the electronic device 100 according to the inventive concepts is not limited thereto.

The electronic device 100 may further include a plurality of sensors (not illustrated) other than the PPG sensor 120. For example, the electronic device 100 may further include an electrocardiogram (ECG) sensor, a motion sensor, and/or the like. The ECG sensor may be configured to generate ECG signals by measuring an ECG of the user. The motion sensor may be configured to continuously measure motions of the user and generate inertial measurement unit (IMU) signals. Sensors that may be included in the electronic device 100 are not limited thereto. In at least one example embodiment, the PPG sensor 120 may further include other bio sensors. For example, the PPG sensor 120 may further include a sensor configured to measure bioimpedance of the user and a sensor configured to sense states or changes in sweat, blood, urine, and/or iris. For example, the PPG sensor 120 may further include a galvanic skin response (GSR) sensor, an electrodermal activity (EDA) sensor, a ballistocardiogram (BCG) sensor, a sweat sensor for sensing hydration or dehydration, an iris sensor, a body temperature sensor, etc.

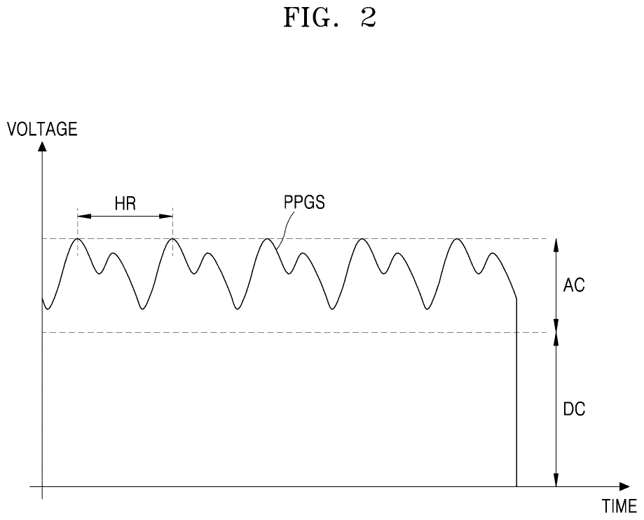

The PPG sensor 120 may be configured to generate PPG signals by measuring pulse waves reflected from the user. The PPG sensor 120 may be configured to measure the pulse waves of the user by using the principle that an amount of light reflected from the user varies according to heartbeats when light is irradiated to the skin of the user. The PPG sensor 120 according to the inventive concepts may be configured to generate a PPG signal (PPGS) by measuring the pulse waves of the user. The PPG signal PPGS may include a signal having a shape according to the pulse waves of the user, and may include a direct current component and an alternating current component. The PPG signal PPGS may be understood with reference to FIG. 2.

In human bodies, light sensitivities, such as ultraviolet-sensitivity, may vary according to skin tones (and/or skin colors). For example, the transmissivity of ultraviolet through skin varies according to the skin tones. For example, when the skin tone is brighter, a greater amount of ultraviolet may enter the skin. On the other hand, when the skin tone is darker, a lesser amount of ultraviolet may enter the skin. Accordingly, it is beneficial to distinguish the skin tone of a user to improve the accuracy of the PPG used in smart watches and/or the like and to determine (e.g., calculate) an amount of ultraviolet absorption of the user.

The PPG sensor 120 according to at least one example embodiment may include a light-emitting module (or a “light-emitter”) configured to emit the light polarized in a first direction, a first light-receiving module (or a “first light-receiver”) configured to receive the light polarized in a second direction different from the first direction, and a second light-receiving module (or a “second light-receiver”) configured to receive light. The electronic device 100 according to the inventive concepts may be configured to distinguish a skin tone of a human body, based on a first PPG signal generated by the first light-receiving module and a second PPG signal generated by a second light-receiving module.

The PPG sensor 120 according to at least some embodiments may include a first light-emitting module configured to emit the light polarized in the first direction, a second light-emitting module configured to emit light, and a light-receiving module configured to receive the light polarized in the second direction different from the first direction. The electronic device 100 according to the inventive concepts may be configured to distinguish the skin tone of the human body based on the first PPG signal and the second PPG signal. In these cases, the first PPG signal is generated based on the light emitted from the first light-emitting module, reflected by the human body, and absorbed by the light-receiving module, and the second PPG signal is generated based on the light emitted from the second light-emitting module, reflected by the human body, and absorbed by the light-receiving module.

The PPG sensor 120 according to at least some embodiments may include a light-emitting module configured to emit the light and a light-receiving module configured to receive the light. The electronic device 100 according to the inventive concepts may be configured to regulate intensity of a current and emit at least two streams of light having difference luminance through the light-emitting module, receive the at least two streams of light, which are reflected by the human body, through the light-receiving module, and distinguish the skin tone of the human body based on a difference between at least two PPG signals generated based on the received light.

The processor 110 may be configured to control general operations of the electronic device 100, and may also be configured to control components, e.g., the PPG sensor 120, the input/output device 130, the communication module 140, the memory 150, the storage 160, and the power module 170. In some embodiments, the processor 110 may include a micro control unit (MCU). However, the processor 110 is not limited thereto, and may also include a processor such as a central processing unit (CPU), a micro processing unit (MPU), a neural processing unit (NPU), and/or the like.

The processor 110 may be configured to process biometric signals received from the PPG sensor 120, e.g., the PPG signal, and monitor health conditions of the user based on the biometric signals.

The processor 110 according to the inventive concepts may be configured to distinguish the skin tone of the user based on the difference between two PPG signals. The processor 110 according to at least one embodiment may be configured to calculate a perfusion index (PI) of each of the at least two PPG signals and distinguish the skin tone of the user based on a difference (e.g., a degree of PI change) between at least two PIs. The processor 110 according to at least one embodiment may be configured to determine (e.g., calculate) a signal-to-noise ratio (SNR) of the direct current components included in each of the at least two PPG signals and distinguish the skin tone of the user based on a difference (e.g., a degree of SNR change) between at least two SNRs. Details of a method of distinguishing the skin tone by the processor 110 will be described later.

The input/output device 130 may include various devices configured to receive user inputs and/or configured to output (e.g., provide) information, notice, and/or the like to the user. The input/output device 130 may include, for example, a display 131 and an audio module 132. The input/output device 130 may further include devices such as a vibration module, an input key, a microphone, and/or the like.

The display 131 may be configured to display various kinds of information based on control by the processor 110. For example, the display 131 may be configured to display biometric information of the user, e.g., a heart rate, an oxygen saturation, and/or the like. The display 131 may be configured to display atrial fibrillation detection information (whether atrial fibrillation has occurred), arrhythmia information (existence of arrhythmia and/or type of arrhythmia), or suspected disease information. The display 131 may also be configured to information requiring motions of the user. The display 131 according to the inventive concepts may be configured to display an amount of vitamin synthesis per hour of the user, based on the distinguished skin tone of the user and the real-time ultraviolet information.

The display 131 may include at least one of various display devices, e.g., liquid crystal displays (LCD), thin-film transistor LCDs (TFT-LCD), organic light-emitting diodes (OLED), light-emitting diodes (LED), active matrix organic LEDs (AMOLED), micro LEDs, mini LEDs, flexible displays, three-dimension displays, and/or the like. In some embodiments, the display 131 may be implemented in the form of a touch screen. In some embodiments, the display 131 may be implemented as an expanded display and/or a flexible display.

The audio module 132 may be configured to output sound, and for example, the audio module 132 may include at least one of an audio codec, a microphone MIC, a receiver, an earphone output, a speaker, and/or the like. The audio module 132 may be configured to output information regarding health state of the user, information regarding symptoms of health conditions of the user, or additional information, in the form of audio signals, based on the biometric information and/or suspected disease information that has been obtained.

The communication module 140 may be configured to communicate with external devices. In some embodiments, the communication module 140 may include a wireless communication module, such as a Bluetooth module, a Wireless Local Area Network (WLAN) such as Wireless Fidelity (Wi-Fi), Wireless Personal Area Network (WPAN), Wireless Universal Serial Bus, Zigbee, Near Field Communication (NFC), and Radio-Frequency Identification (RFID), communication interfaces configured to access mobile cellular networks (such as 3rd Generation (3G), 4th Generation (4G), Long Term Evolution (LTE), and/or the like), etc. In some embodiments, the communication module 140 may further include a communication interface configured to access a wired NFC network.

The communication module 140 may be configured to transmit, to an external electronic device (e.g., a smart phone of the user), the information regarding the physical state of the user, the information regarding symptoms of the health state of the user, or the additional information. The communication module 140 may be configured to receive real-time ultraviolet information according to a current position of the user. The electronic device 100 according to the inventive concepts may be configured to calculate an amount of real-time vitamin synthesis based on the real-time ultraviolet information and information regarding the distinguished skin tone of the user.

The memory 150 may be implemented as a volatile memory such as dynamic random access memory (DRAM) or static RAM (SRAM) or a nonvolatile resistive memory such as phase change RAM (PRAM) or resistive RAM (ReRAM). In embodiments, the memory 150 may be integrated into the processor 110.

Operation programs, applications programs, and/or the like executed by the processor 110 may be loaded in the memory 150 for execution. For example, programs including instructions for implementing the aforementioned functions of the processor 110 may be loaded to the memory 150 and executed by the processor 110.

In addition, the memory 150 may be configured to store data to be processed by the processor 110 or generated in the processor 110. For example, the memory 150 may be configured to temporarily store records about biometric information measurement (e.g., the number of times of measurement, a time period of measurement), the biometric information of the user, information about suspected diseases, and/or the like.

The storage 160 may be implemented as a nonvolatile memory device such as a NAND flash, a resistive memory, and/or the like, and for example, may be provided in the form of a memory card (e.g., a multimedia card (MMC), an eMMC, a secure digital (SD) card, and a micro SD card). The storage 160 may be configured to store the data generated by the processor 110. The storage 160 may be configured to store the record about biometric information measurement (e.g., the number of times of measurement, the time period of measurement), the biometric information of the user, the information about suspected diseases, and/or the like).

The power module 170 may include a battery, a charge circuit, a power management unit (PMU), and/or the like. In some embodiments, the PMU may be integrated into the processor 110. The power module 170 may be configured to generate and provide power sources used in the electronic device 100, based on the power provided from the battery and/or an external power source. The power module 170 may also be configured to charge the battery based on the external power source. The PMU may be configured to manage the power provided to the components. For example, the PMU may be configured to provide the power sources to the components and adjust a level of the power source (e.g., a voltage level) provided to the components or an operation frequency, based on an operation state of the electronic device 100 or an operation state of each component. In addition, the PMU may also be configured to block the power.

FIG. 2 is a graph showing the PPG signal according to at least one embodiment.

Referring to FIG. 2, the PPG signal PPGS may include an alternating current component (AC) and a direct current component (DC). The PPG signal PPGS may have periodicity according to a heart rate (HR) of the user.

In the inventive concepts, the direct current component DC of the PPG signal PPGS may refer to a component constantly maintained regardless of a pulse of the user. As described above, a thickness of a blood vessel of the user may vary according to the pulse of the user, and the PPG sensor 120 (see FIG. 1) may be configured to generate the PPG signal PPGS by measuring the pulse waves of the user. The PPG sensor 120 (see FIG. 1) may be configured to measure the pulse waves of the user by using a principle in which an amount of light adsorption changes due to change in the thickness of the blood vessel according to heartbeat when the light is irradiated to the skin of the user. Accordingly, the direct current component DC of the PPG signal PPGS may include a component regardless of the pulse waves (e.g., a component corresponding to a thickness of the blood vessel when the blood vessel of the user is not expanded and/or a component generated by the light reflected by and returned from a surface of the skin of the user). Therefore, PPG may be used for measuring the pulse of the user, and PPG performance may be determined according to the alternating current component AC (or “pulsatile” component) corresponding to the change in the thickness of the blood vessel, rather than according to the direct current component DC.

In the inventive concepts, the alternating current component AC of the PPG signal PPGS may indicate a component in which an amount of light absorbed by the electronic device 100 (see FIG. 1) varies according to the heart rate of the user.

A degree of change in the thickness of the blood vessel due to the pulse of the user may be less than an original thickness of the blood vessel, and there may be light reflected from the surface of the skin of the user regardless of the measurement on the pulse of the user. Accordingly, the alternating current component AC of the component included in the PPG signal PPGS may be less than the direct current component DC included in the PPG signal PPGS. As information needed for measuring the pulse of the user is the alternating current component AC included in the PPG signal PPGS, the PPG performance may be improved as the intensity of the alternating current component AC increases with respect to the direct current component DC. Accordingly, to improve the PPG performance, it is required to reduce the amount of light reflected from the surface of the skin and absorbed into the electronic device.

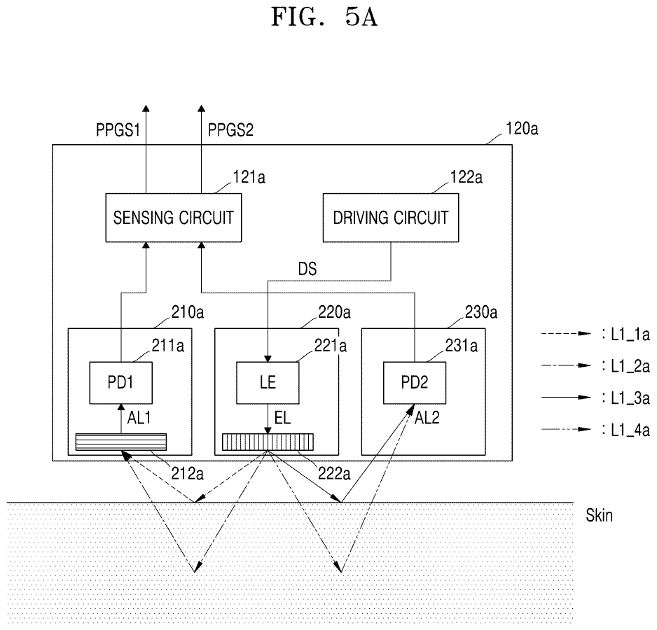

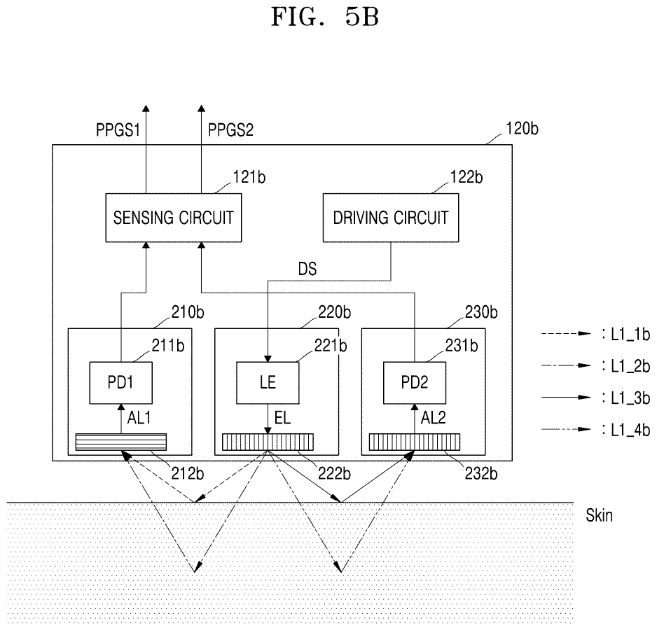

The electronic device 100 (see FIG. 1) according to the inventive concepts may include a polarizer to increase the intensity of the alternating current component AC with respect to the direct current component DC. For example, the electronic device 100 (see FIG. 1) may include the polarizer for improvement of the PPG performance. Details thereof will be described later with reference to FIG. 5A and thereafter. The electronic device according to the inventive concepts may be configured to distinguish the skin tone of the user, based on a degree of improvement in properties of the PPG signal by the polarizer. As described above, the amount of light absorbed may vary according to the skin tone of the user, and accordingly, a degree of improvement in the properties of the PPG signal (e.g., the PI to be described later or the SNR in the alternating current component) may also vary. The electronic device according to the inventive concepts may be configured to distinguish the skin tone of the user by using a property that the degree of improvement in the properties of the PPG signal varies according to the skin tone.

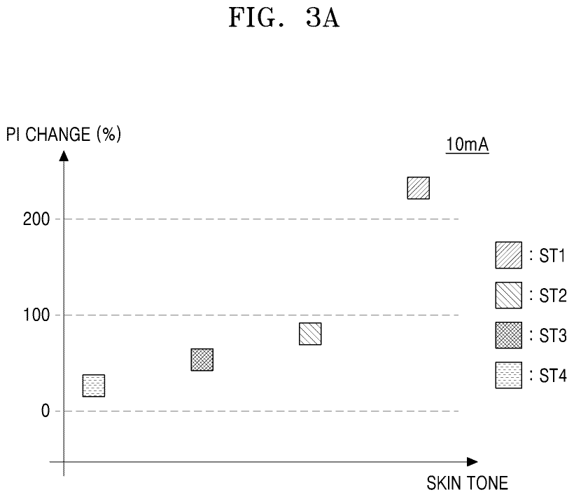

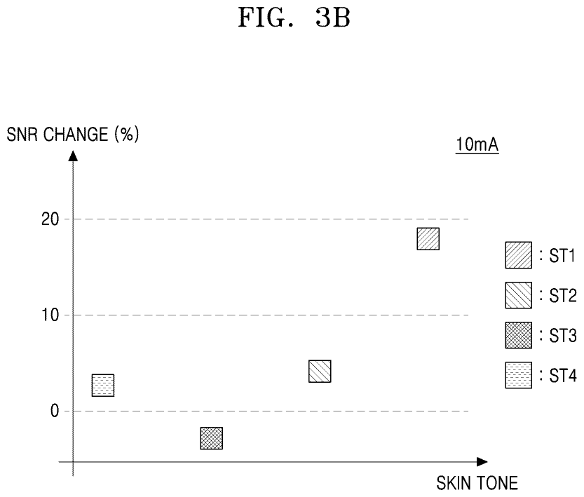

FIGS. 3A and 3B are graphs showing changes in the PPG performance according to at least one embodiment.

FIGS. 3A and 3B are graphs showing changes in the PPG performance when a value of a driving current for driving the light-emitting element included in the light-emitting module is 10 mA.

FIG. 3A illustrates a comparison of PI changes when the polarizer is used compared with the polarizer is not used. PI is a value indicating a ratio of the alternating current component to the direct current component included in the PPG signal. As described above, when the ratio of the alternating current component to the direct current component is great, the PPG performance may be improved, and it may be understood that a greater rate of the PI change in FIG. 3A indicates greater improvement in the performance of PPG. As described above, as a rate of light absorption may vary according to the skin tone, the rate of PI change may vary according to the skin tone.

Referring to FIG. 3A, the skin tone may become brighter from a first skin tone ST1 to a fourth skin tone ST4. For example, the first skin tone ST1 may be darker than a second skin tone ST2, the second skin tone ST2 may be darker than a third skin tone ST3, and the third skin tone ST3 may be darker than the fourth skin tone ST4. Referring to FIG. 3A, as the skin tone becomes darker, the rate of PI change may increase. For example, a degree of improvement in PI may decrease from the first skin tone ST1 to the fourth skin tone ST4. However, the degree of improvement in PI and the number of skin tones according to the inventive concepts are only examples and are not limited to the descriptions given above or hereinafter.

Accordingly, the electronic device according to the inventive concepts may be configured to distinguish the skin tone of the user based on the rate of PI change (or the degree of improvement of PI).

FIG. 3B illustrates a comparison of SNR changes of the direct current component when the polarizer is used compared with the polarizer is not used. As described above, when the ratio of the alternating current component to the direct current component is great, the PPG performance may be improved, and it may be understood that a greater rate of the SNR change in FIG. 3B indicates greater improvement in the performance of PPG. As described above, as the rate of light absorption may vary according to the skin tone, the rate of SNR change may vary according to the skin tone.

Accordingly, the electronic device according to the inventive concepts may be configured to distinguish the skin tone of the user based on the rate of SNR change (or the degree of improvement of SNR).

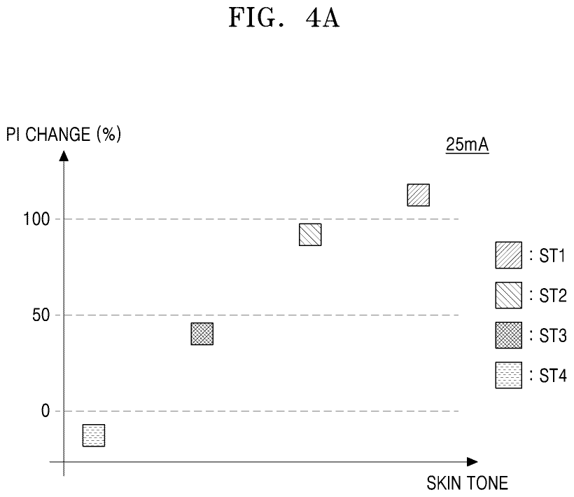

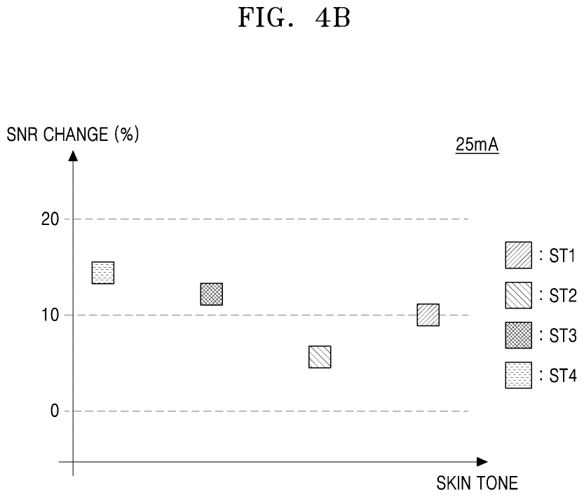

FIGS. 4A and 5B are diagrams each illustrating change in the PPG performance according to at least one embodiment.

FIGS. 4A and 4B are graphs each showing changes in the PPG performance when a value of a driving current for driving the light-emitting element included in the light-emitting module is 25 mA. FIG. 4A may be understood with reference to FIG. 3A, and FIG. 4B may be understood with reference to FIG. 3B. Accordingly, same descriptions as given above will not be repeatedly given.

FIG. 4A illustrates a comparison of PI changes when the polarizer is used compared with when the polarizer is not used. Referring to FIG. 4A, as the skin tone becomes darker, the degree of PI change may increase, like in FIG. 3A. For example, the degree of improvement in PI may decrease from the first skin tone ST1 to the fourth skin tone ST4. However, the degree of improvement in PI and the number of skin tones according to the inventive concepts are only examples and are not limited to the descriptions given above and hereinafter.

FIG. 4B illustrates a comparison of SNR changes of the direct current component when the polarizer is used compared with when the polarizer is not used. FIG. 4B may be understood from the descriptions given above with reference to FIG. 3B.

The degree of change in the PPG performance may vary according to the intensity of the driving current for driving the light-emitting device, as illustrated in FIGS. 3A to 4B.

FIGS. 5A and 5B are block diagrams respectively illustrating PPG sensors 120a and 120b according to some embodiments.

Referring to FIG. 5A, the PPG sensor 120a may include a sensing circuit 121a, a driving circuit 122a, a first light-receiving module 210a, a light-emitting module 220a, and a second light-receiving module 230a. The light-emitting module 220a may include at least one light-emitting element 221a. For example, the light-emitting module 220a may include a light-emitting diode (LED). The first light-receiving module 210a and the second light-receiving module 230a may respectively include at least one light-receiving elements (e.g., a first light-receiving element 211a and a second light-receiving element 231a). For example, the first light-receiving module 210a and the second light-receiving module 230a may each include an optical sensor including a photodetector.

For convenience of explanation, in the inventive concepts, it is illustrated that the emission module includes a luminous element and the light-receiving module includes a light-receiving element. However, the inventive concepts are not limited thereto, and the numbers of the luminous element and the light-receiving element may be modified. In addition, for convenience of explanation, the embodiments may be described later under assumption that the light-receiving elements 211a and 231a include photodiodes and the light-emitting element 221a includes an LED, but the inventive concepts are not limited thereto.

Referring to FIG. 5A, the driving circuit 122a may be configured to provide a driving current DS to the light-emitting element 221a. For example, the driving circuit 122a may be configured to provide the driving current DS to the LEDs. The driving circuit 122a may include a metal-oxide silicon field-effect transistor (MOSFET) and a digital-analog converter for controlling currents.

The sensing circuit 121a may be configured to convert reflected light AL1 and AL2 measured (or received) by the light-receiving elements 211a and 231a into PPG signals (e.g., a first PPG signal PPGS1 and a second PPG signal PPGS2). More particularly, the first light-receiving element 211a may be configured to output a voltage level, which corresponds to the reflected light AL1 received by the first light-receiving element 211a, to the sensing circuit 121a, and the sensing circuit 121a may be configured to generate a first PPG signal PPGS1, based on the voltage level corresponding to the reflected light AL1. The shape of the PPG signal may be understood with reference to FIG. 2. Similarly, the second light-receiving element 231a may be configured to a voltage level, which corresponds to the reflected light AL2 received by the second light-receiving element 231a, to the sensing circuit 121a, and the sensing circuit 121a may be configured to generate a second PPG signal PPGS2, based on the voltage level corresponding to the reflected light AL2. The sensing circuit 121a may include an amplifier, a filter, and an analog-digital converter. For example, the amplifier may be implemented as a transimpedance amplifier. The sensing circuit 121a may be configured to convert measured reflected light into a voltage signal by using the amplifier and filter the voltage signal by using a low pass filter. For example, the low pass filter may be configured to block a frequency component having a value greater than 5 Hz. In the inventive concepts, the reflected light AL1 and AL2 received by the first light-receiving element 211a and the second light-receiving element 231a may be referred to as absorbed light for distinction from reflected light reflected from the skin.

The PPG sensor 120a may be configured to continuously (e.g., repeatedly, as long as the user and/or host does not stop) measure the pulse waves of the user and generate the first PPG signal PPGS1 and the second PPG signal PPGS2. The light-emitting module 220a, the first light-receiving module 210a, and the second light-receiving module 230a of the PPG sensor 120a may be in contact with the skin of the user, and accordingly, the PPG sensor 120a may always measure the pulse waves of the user.

The light-emitting module 220a according to the inventive concepts may be configured to receive the driving current DS from the driving circuit 122a and emit light. More particularly, the light-emitting element 221a included in the light-emitting module 220a may be configured to emit light based on the driving current DS. In the invented concept, for convenience of explanation, light emitted from the light-emitting element 221a is referred to as emitted light EL. The light-emitting module 220a according to the inventive concepts may further include a first polarizer 222a. The polarizer may include a polarization film (e.g., poly-vinyl-alcohol (PVA)). The first polarizer 222a may polarize light in a first direction. As the first polarizer 222a only transmits light in a certain direction, light (the emitted light EL) transmitted through the first polarizer 222a may vibrate only in a first direction. In the inventive concepts, the term “first direction” is used to distinguish from a second direction to be described later, and the first polarizer 222a, which polarizes light in the first direction, does not necessarily indicate a linear polarizer which linearly polarizes light. For example, the first polarizer 222a may also include a circular polarizer or an elliptical polarizer. However, in the inventive concepts, for description, the following descriptions will be given under a premise that the first polarizer is a linear polarizer.

As described above, the PPG sensor 120a may be configured to emit light polarized in the first direction through the first polarizer 222a. The light polarized in the first direction may be emitted toward the skin of the user, and may be received through the first light-receiving module 210a or the second light-receiving module 230a along a first light path L1_1a to a fourth light path L1_4a.

The first light-receiving module 210a according to the inventive concepts may be configured to receive light along the first light path L1_1a and the second light path L1_2a. The light reflected from the surface of the skin of the user may maintain original properties (e.g., polarization properties). However, the original properties (e.g., a polarization direction) of the light reflected from the skin of the user may change due to various scattering media (e.g., blood, skin tissues, and bones). Accordingly, the light received by the first light-receiving module 210a along the first light path L1_1a may be the light polarized in the first direction. On the other hand, light received by the first light-receiving module 210a along the second light path L1_2a may be light that lost the polarization property of being polarized in the first direction. The first light-receiving module 210 according to the inventive concepts may include a second polarizer 212a polarized in a second direction different from the first direction. For example, the second direction may be perpendicular to the first direction, and there is possibility that the light polarized in the first direction may not be transmitted through the second polarizer 212a. However, the inventive concepts are not limited to the aforementioned example, and the second direction may include a direction that is not perpendicular to the first direction but different to the first direction. For example, the second polarizer 212a may transmit only a portion of light polarized in the first direction. Hereinafter, for convenience of description, the description will be given under a premise that the first direction and the second direction are perpendicular to each other.

The first light-receiving element 211a according to the inventive concepts may receive light transmitted through the second polarizer 212a. The light along the first light path L1_1a, e.g., the light reflected from the surface of the skin of the user, maintains the property of being polarized in the first direction, and thus may be not transmitted through the second polarizer 212a. Accordingly, the light reflected from the surface of the skin of the user may be not received by the first light-receiving element 211a. Accordingly, in the PPG signal described above with reference to FIG. 2, an occupation of the direct current component may decrease. The light along the second light path L1_2a, e.g., light reflected from inside the skin of the user, may lose the property of being polarized in the first direction, and thus, may be partially transmitted through the second polarizer 212a. Accordingly, in the PPG signal described with reference to FIG. 2, an occupation of the alternating current component may increase. Accordingly, the PPG sensor 120a may be configured to generate the first PPG signal PPGS1 corresponding to an amount of the light received by the first light-receiving element 211a, based on the first polarizer 222a and the second polarizer 212a.

The second light-receiving module 230a according to the inventive concepts may be configured to receive light along the third light path L1_3a and the fourth light path L1_4a. As described above, the light reflected from the surface of the skin of the user may maintain the original polarization properties, and the polarization properties of the light reflected from inside the skin of the user may change due to various scattering media inside the skin. Accordingly, the light received by the second light-receiving module 230a along the third light path L1_3a may include the light polarized in the first direction. On the other hand, light received by the second light-receiving module 230a along the fourth light path L1_4a may include light that lost the polarization property of being polarized in the first direction. Unlike the first light-receiving module 210a, the second light-receiving module 230a according to the inventive concepts may not include a polarizer. Accordingly, the PPG sensor 120a may be configured to generate the second PPG signal PPGS2 corresponding to an amount of light received by the second light-receiving element 231a.

A distance between the first light-receiving module 210a and the light-emitting module 220a may be equal to a distance between the second light-receiving module 230a and the light-emitting module 220a. Although the first light-receiving module 210a and the second light-receiving module 230a are apart from the light-emitting module 220a in the same distance, according to whether a polarizer is included or not, the first PPG signal PPGS1 and the second PPG signal PPGS2 may be different from each other. As described above, an amount of light being absorbed may vary according to the skin tone of the user, and therefore, a difference between the first PPG signal PPGS1 and the second PPG signal PPGS2 may vary according to the skin tone of the user. For example, referring to FIG. 3A, as the skin tone becomes darker (that is, from the fourth skin tone ST4 to the first skin tone ST1, a PI of the first PPI signal PPGS1 (hereinafter, may be referred to as a first PI) to a PI of the second PPG signal PPGS2 (hereinafter, may be referred to as a second PI) may increase. The electronic device 100 (see FIG. 1) according to the inventive concepts may be configured to distinguish the skin tone of the user based on the difference between the first PPG signal PPGS1 and the second PPG signal PPGS2. For example, referring to FIG. 3A, a value of the driving current DS may be 10 mA, and the electronic device 100 (see FIG. 1) according to the inventive concepts may distinguish the skin tone of the user as the first skin tone ST1 (see FIG. 3A) when a ratio of increase in the first PI of the first PPG signal to the second PI of the second PPG signal PPGS2 is 200% or greater. Referring to FIG. 3B, the driving current DS may have a value of 10 mA, and the electronic device 100 (see FIG. 1) according to the inventive concepts may distinguish the skin tone of the user as the second skin tone ST2 (see FIG. 3B) when a ratio of increase in a SNR of the alternating current component included in the first PPG signal (hereinafter, may be referred to as a fist SNR) to a SNR of the alternating current included in the second PPG signal PPGS2 (hereinafter, may be referred to as a second SNR) is less than 0%.

The PPG sensor 120a may be configured to deliver the first PPG signal PPGS1 and the second PPG signal PPGS2 to the processor 110 (see FIG. 1). For example, transmission of the PPG signals may be performed based on a Serial Peripheral Interface (SPI)-based interface. However, the inventive concepts is not limited thereto, and at least one interface method among High Speed Serial Interface (HSSI) methods Inter-Integrated circuit (I2C), Mobile Industry Processor Interface (MIPI), Universal Asynchronous Receiver/Transmitter (UART) may be applied between the PPG sensor 120a and the processor 110 (see FIG. 1). The processor 110 (see FIG. 1) according to the inventive concepts may be configured to distinguish the skin tone of the user based on the difference between the first PPG signal and the second PPG signal. For example, as described above, the processor 110 (see. FIG. 1) may be configured to receive the first PPG signal and the second PPG signal, calculate the PIs or SNRs and/or the like of the first PPG signal and the second PPG signal, and distinguish the skin tone of the user based on a difference between calculation results.

Similarly to the PPG sensor 120a described above with reference to FIG. 5A, the PPG sensor 120b illustrated in FIG. 5B may include a sensing circuit 121b, a driving circuit 122b, a first light-receiving module 210b, a light-emitting module 220b, and a second light-receiving module 230b. The light-emitting module 220b may include a light-emitting element 221b and a first polarizer 222b, and the first light-receiving module 210b may include a first light-receiving element 211b and a second polarizer 212b. The second light-receiving module 230b may include a second light-emitting element 231b. However, unlike the PPG sensor 120a illustrated in FIG. 5A, the PPG sensor 120b illustrated in FIG. 5B may further include a third polarizer 232b. More particularly, unlike the second light-receiving module 230a, the second light-receiving module 230b illustrated in FIG. 5B may further include the third polarizer 232b. FIG. 5B may be understood with reference to FIG. 5A, and hereinafter, same descriptions will not be repeatedly given.

Referring to FIG. 5B, the second light-receiving module 230b according to the inventive concepts may include the third polarizer 232b which polarizes light in the first direction. That is, the second polarizer 212b and the third polarizer 232b may include polarizers which polarize the light in the same direction. The second light-receiving module 230b may only receive the light polarized in the first direction through the third polarizer 232b. Referring to FIG. 5B, the first light-receiving module 210b may not receive the light along a first light path L1_1b and receive a portion of light along a second light path L1_2b. The second light-receiving module 230b may receive light along a third light path L1_3b but receive only a portion of light along a fourth light path L1_4b. Accordingly, the second light-receiving module 230b corresponding to the light received by the second light-receiving module 230b and the first PPG signal PPGS1 corresponding to the light received by the first light-receiving module 210b may be different from each other. For example, the electronic device 100 (see FIG. 1) according to the inventive concepts may be configured to distinguish the skin tone of the user based on a rate of PI change (or a difference) of the first PPG signal PPGS1 corresponding to the light received by the first light-receiving module 210b to the PI of the second PPG signal PPGS2 corresponding to the light received by the second light-receiving module 230b. For example, the electronic device 100 (see FIG. 1) according to the inventive concepts may be configured to distinguish the skin tone of the user, based on a rate of change (or a difference) in the SNR of the alternating current component included in the first PPG signal PPGS1 to the SNR of the alternating current component included in the second PPG signal PPGS2.

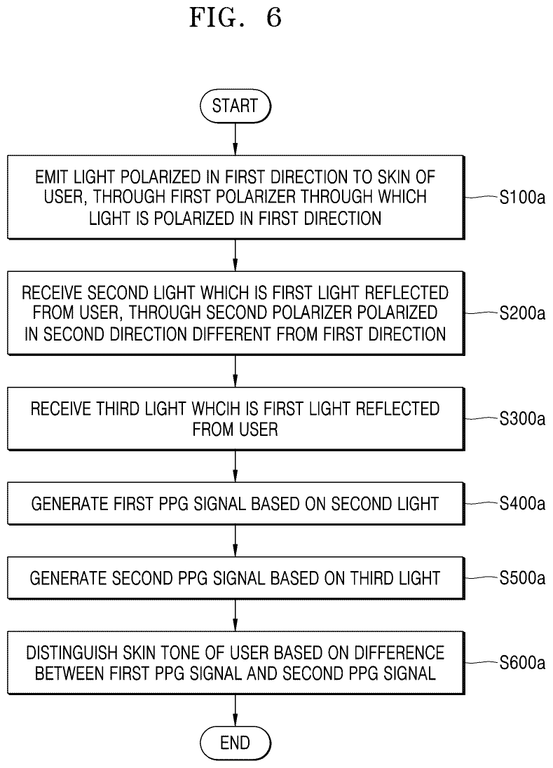

FIG. 6 is a flowchart for describing an operating method of the electronic device, according to at least one embodiment.

FIG. 6 is a flowchart for describing the operating method of the electronic device described above with reference to FIGS. 5A and 5B. Accordingly, FIG. 6 may be understood with reference to FIGS. 5A and 5B.

Referring to FIG. 6, in S100a, the electronic device emits first light polarized in the first direction to the skin of the user, through the first polarizer polarized in the first direction. In FIGS. 5A and 5B, the first light may indicate light emitted from the first light-emitting module 220a and the second light-emitting module 220b and not yet reflected from the surface of the skin of the user or inside the skin of the user.

In S200a, the electronic device receives second light obtained by reflection of the first light from the user, through the second polarizer polarized in the second direction different from the first direction. The first direction and the second direction may be different from each other. For example, the first direction may be a direction perpendicular to the second direction. In FIGS. 5A and 5B, the second light may indicate light that is the first light reflected from the surface of the skin of the user or inside the skin of the user and received by the first-receiving modules 210a and 210b.

In S300a, the electronic device receives third light that is the first light reflected from the user. As described above with reference to FIG. 5B, the electronic device according to the inventive concepts may receive the third light polarized in the first direction, through the third polarizer polarized in the first direction. In FIGS. 5A and B, the third light may indicate light that is the first light reflected from the surface of the skin of the user or inside the skin of the user and received by the second light-receiving modules 230a and 230b. S200a and S300a may occur in parallel and/or may occur in series.

In S400a, the electronic device generates the first PPG signal based on the second light.

In S500a, the electronic device generates the second PPG signal based on the third light. As described above with reference to FIG. 5A, the first PPG signal is generated based on the light received through the polarizer, and thus the PI of the first PPG signal may be greater than the PI of the second PPG signal. S400a and S500a may occur in parallel and/or may occur in series.

In S600a, the electronic device may distinguish the skin tone of the user, based on the difference between the first PPG signal and the second PPG signal. For example, the electronic device may distinguish the skin tone of the user, based on the difference between the first PI of the first PPG signal and the second PI of the second PPG signal (see FIGS. 3A and 4A). The electronic device according to the inventive concepts may distinguish the skin tone of the user as a dark skin tone as the first PI to the second PI increases. The electronic device according to the inventive concepts may distinguish the skin tone of the user, based on a difference between a first signal-to-noise ratio (SNR) of the alternating current component included in the first PPG signal to the second SNR of the alternating current component included in the second PPG signal (see FIGS. 3B and 4B). In at least some embodiments, the operations of the electronic device may be adjusted based on the skin tone of the user. For example, the intensity of the light-emitting module and/or the sensitivity of the light-receiving modules may be adjusted based on the determined skin tone. Additionally, the electronic device may compensate for the differences in the ratio between alternating current component AC and direct current component DC due to skin tone in operations applying the ratio between alternating current component AC and direct current component DC (e.g., determining oxygen saturation) wherein differences in skin tone may otherwise cause inaccuracies.

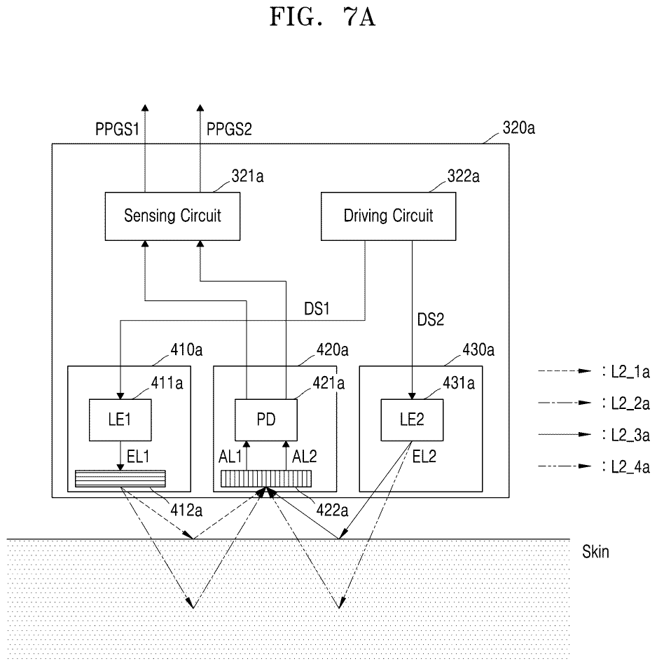

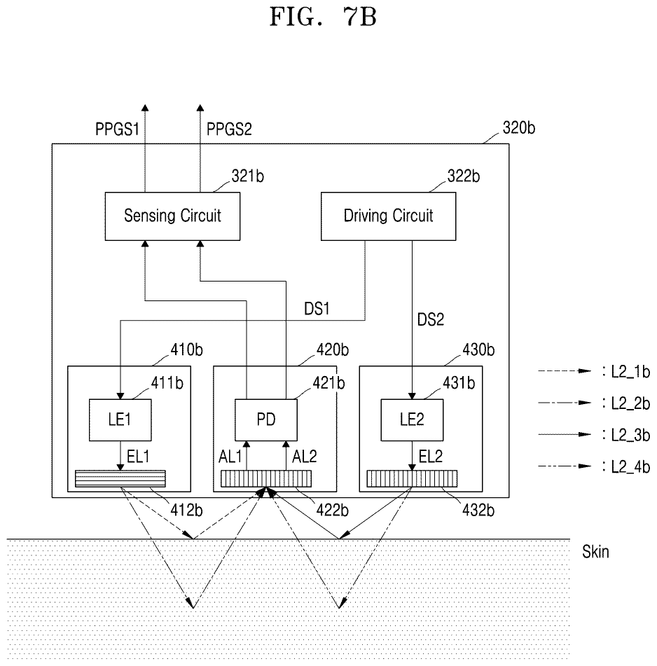

FIGS. 7A and 7B are block diagrams illustrating PPG sensors 320a and 320b according to at least one embodiment.

FIGS. 7A and 7B may be understood based on the description given above with reference to FIGS. 5A and 5B. Therefore, hereinafter, repeated descriptions will not be given.

Referring to FIG. 7A, the PPG sensor 320a may include a sensing circuit 321a, a driving circuit 322a, a first light-emitting module 410a, a light-receiving module 420a, and a second light-emitting module 430a. The light-receiving module 420a may include a light-receiving element 421a and a first polarizer 422a polarized in the first direction, the first light-emitting module 410a may include a first light-emitting element 411a and a second polarizer 412a polarized in the second direction different from the first direction, and the second light-emitting module 430a may include a second light-emitting element 431a. The PPG sensor, the sensing circuit, the driving circuit, the light-emitting element, the light-receiving element, and the polarizer have been described with reference to FIGS. 5A and 5B, and same descriptions will not be repeatedly given.

Referring to FIG. 7A, the first light-emitting module 410a may be configured to receive a first driving circuit DS1 from the driving circuit 322a and emit first emitted light EL1. More particularly, the first light-emitting element 411a included in the first light-emitting module 410a may be configured to emit the first emitted light EL1 based on the first driving current DS1. The first light-emitting module 410a may be configured to emit the light polarized in the second direction, through the second polarizer 412a polarized in the second direction. The light may be received by the light-receiving module 420a along a first light path L1 and a second light path L2.

The second light-emitting module 430a may be configured to receive a second driving current DS2 from the driving circuit 322a and emit second emitted light EL2. More particularly, the second light-emitting element 431a included in the second light-emitting module 430a may be configured to emit the second emitted light EL2 based on the second driving current DS2. The second emitted light EL2 may be received by the light-receiving module 420a along a third light path L2_3a and a fourth light path L2_4a.

The first light-emitting module 410a and the second light-emitting module 430a may emit the emitted light in different periods, respectively. In other words, periods in which the second driving current DS2 and the first driving current DS1 are output may be different. For example, the second light-emitting module 430a according to the inventive concepts may emit the second emitted light EL2 after the light polarized in the second direction, which is emitted from the first light-emitting module 410a, is received by the light-receiving module 420a. However, this is only an example, and the inventive concepts are not limited thereto. For example, the first light-emitting module 410a according to the inventive concepts may emit the first emitted light EL1 after the light, which is emitted from the second light-emitting module 430a, is received by the light-receiving module 420a.

The light-receiving module 420a may receive light emitted from the first light-emitting module 410a and the second light-emitting module 430a and reflected from the skin of the user, through the light-receiving element 421a and the first polarizer 422a in which the light is polarized in the second direction different from the first direction.

The PPG sensor 320a according to the inventive concepts may receive the light along a first light path L2_1a and a second light path L2_2a through the light-receiving module 420a and generate the first PPG signal PPGS1 corresponding to an amount of the light received. As described above with reference to FIG. 5A, the first direction and the second direction may be perpendicular to each other. Accordingly, the light along the first light path L2_1a may maintain the property of being polarized in the second direction, and thus, the light along the first light path L2_1a may be not received by the light-receiving element 421a. However, as described above, the first direction and the second direction according to the inventive concepts are not limited to being perpendicular to each other. As the light along the second light path L2_2a may be reflected from inside the skin of the user and lose the original polarization property, and therefore may be transmitted through the first polarizer 422a and received by the light-receiving element 421a. For example, most of the first absorbed light AL1 may include the light reflected from inside the skin of the user, like the light along the second light path L2_2a.

The PPG sensor 320a according to the inventive concepts may receive the light along a third light path L2_3a and a fourth light path L2_4a through the light-receiving module 420a and generate the second PPG signal PPGS2 corresponding to an amount of the light received. For example, the second absorbed light AL2 may include the light along the third light path L2_3a and the fourth light path L2_4a. Accordingly, the first PPG signal PPGS1 and the second PPG signal PPGS2 may be different from each other. For example, the PI of the first PPG signal PPGS1 may be greater than the PI of the second PPG signal PPGS2. The electronic device 100 (see FIG. 1) according to the inventive concepts may be configured to distinguish the skin tone of the user based on the difference between the first PPG signal PPGS1 and the second PPG signal PPGS2. For example, referring to FIG. 3A, a value of the first driving current DS1 and the second driving current DS2 may be 10 mA, and when a ratio of change (or an increase rate) in the PI of the first PPG signal PPGS1 to the PI of the second PPG signal PPGS2 is 200% or greater, the electronic device may determine that the skin tone of the user is the first skin tone ST1 (see FIG. 3A). For example, referring to FIG. 3B, a value of the driving current DS may be 10 mA, and the electronic device 100 (see FIG. 1) according to the inventive concepts may distinguish the skin tone of the user as the second skin tone ST2 (see FIG. 3B) when a ratio of increase in the SNR of the alternating current component included in the first PPG signal PPGS1 to the SNR of the alternating current component included in the second PPG signal PPGS2 is less than 0%.

Referring to FIG. 7B, the PPG sensor 320b may include a sensing circuit 321b, a driving circuit 322b, a first light-emitting module 410b, a light-receiving module 420b, and a second light-emitting module 430b. The light-receiving module 420b may include a light-receiving element 421b and a first polarizer 422b polarized in the first direction, the first light-emitting module 410b may include a first light-emitting element 411b and a second polarizer 412b polarized in the second direction different from the first direction, and the second light-emitting module 430b may include a second light-emitting element 431b. However, the PPG sensor 320b illustrated in FIG. 7B may further include a third polarizer 432b, unlike the PPG sensor 320a illustrated in FIG. 7A. More particularly, the second light-emitting module 430b illustrated in FIG. 7B may further include the third polarizer 432b, unlike the second light-emitting module 430a illustrated in FIG. 7A. FIG. 7B may be understood with reference to FIG. 7A, and hereinafter, same descriptions will not be repeatedly given.

Referring to FIG. 7B, the second light-emitting module 430b according to the inventive concepts may include the third polarizer 432b polarized in the first direction. That is, the first polarizer 422b and the third polarizers 432b may be polarizers polarized in the same direction. The light-receiving module 420b may only receive the light polarized in the first direction through the first polarizer 422b. Accordingly, in FIG. 7B, the light along a second light path L2_2b may lose the property of being polarized in the second direction and thus may be partially received by the light-receiving module 420b. On the other hand, the light along a first light path L2_1b may maintain the property of being polarized in the second direction and thus may be not received by the light-receiving module 420b. Accordingly, most of the first absorbed light AL1 may include light along the second light path L2_2b. In FIG. 7B, light along a fourth light path L2_4b may lose the property of being polarized in the first direction, and accordingly, most of the second absorbed light AL2 may include light along a third light path L2_3b. Therefore, a difference between the first PPG signal PPGS1 and the second PPG signal PPGS2 with reference to FIG. 7B may be greater than a difference between the first PPG signal PPGS1 and the second PPG signal PPGS2 with reference to FIG. 7A. However, the inventive concepts are not limited thereto. The electronic device 100 (see FIG. 1) according to the inventive concepts may distinguish the skin tone of the user based on the difference between the first PPG signal PPGS1 and the second PPG signal PPGS2.

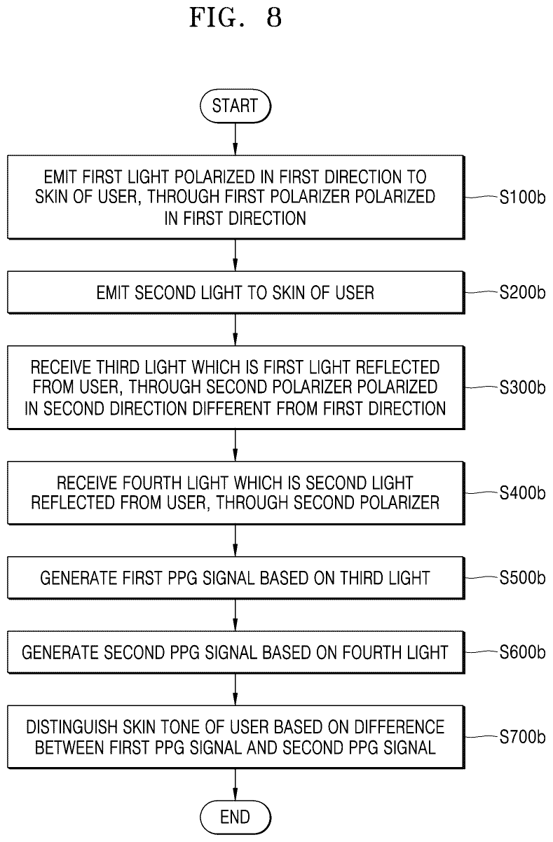

FIG. 8 is a flowchart for describing an operating method of the electronic device, according to at least one embodiment.

FIG. 8 is a flowchart for describing the operating method of the electronic device described above with reference to FIGS. 7A and 7B. Accordingly, FIG. 8 may be understood with reference to FIGS. 7A and 7B.

Referring to FIG. 8, in S100b, the electronic device emits the first light polarized in the first direction to the skin of the user, through the first polarizer polarized in the first direction.

In S200b, the electronic device emits second light to the skin of the user.

In S300b, the electronic device receives third light obtained by reflection of the first light from the user, through the second polarizer polarized in the second direction different from the first direction.

In S400b, the electronic device receives fourth light that is the second light reflected from the user, through the second polarizer.

In S500b, the electronic device generates the first PPG signal based on the third light.

In S600b, the electronic device generates the second PPG signal based on the fourth light.

In S700b, the electronic device distinguishes the skin tone of the user, based on the difference between the first PPG signal and the second PPG signal.

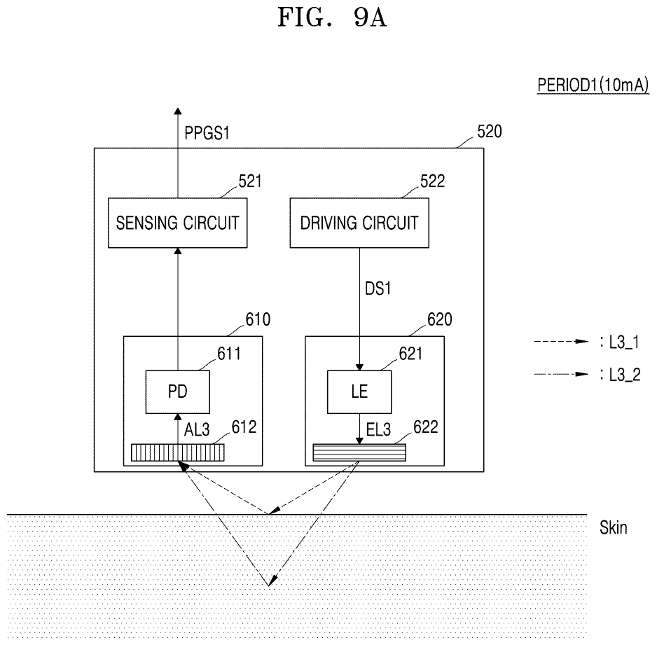

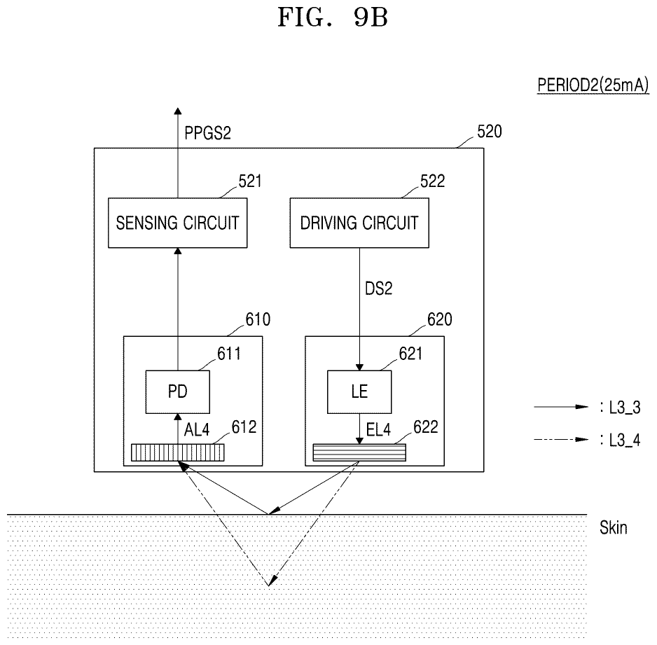

FIGS. 9A and 9B are block diagrams illustrating a PPG sensor 520 according to at least one embodiment.

FIGS. 9A and 9B may be understood based on the description given above with reference to FIGS. 5A and 5B. Therefore, hereinafter, repeated descriptions will not be given.

Referring to FIGS. 9A and 9B, the PPG sensor 520 may include a sensing circuit 521, a driving circuit 522, a light-receiving module 610, and a light-emitting module 620. The light-receiving module 610 may include a light-receiving element 611 and a first polarizer polarized in the first direction, and the light-emitting module 620 may include a light-emitting element 621 and a second polarizer 622 polarized in the second direction different from the first direction. The PPG sensor, the sensing circuit, the driving circuit, the light-emitting element, the light-receiving element, and the polarizer have been described with reference to FIGS. 5A and 5B, and same descriptions will not be repeatedly given.

Referring to FIG. 9A, in a first period PERIOD1, the PPG sensor 520 may be configured to generate the first PPG signal PPGS1 based on the first driving current DS1. The first driving current DS1 may be 10 mA. Referring to FIG. 9B, in a second period PERIOD2, the PPG sensor 520 according to the inventive concepts may generate the second PPG signal PPGS2 based on the second driving current DS2. The second driving current DS2 may be 25 mA. The light-emitting element 621 may emit light proportional to the intensity of the driving current. Accordingly, intensity of second emitted light EL4 emitted by the light-emitting element 621 in the second period PERIOD2 is greater than the intensity of first emitted light EL3 emitted by the light-emitting element 621 in the first period PERIOD1.

The light-emitting module 620 may emit the light through the second polarizer 622 polarized in the second direction. Accordingly, the light emitted from the light-emitting module 620 may include light polarized in the second direction. The light polarized in the second direction may be reflected from the surface of the skin or inside the skin and received by the light-receiving module 610.

Referring to FIG. 9A, in the first period PERIOD1, the light-receiving module 610 may receive light along a first light path L3_1 and a second light path L3_2. The light-receiving module 610 may absorb light through the first polarizer 612 polarized in the first direction different from the second direction. Accordingly, as described above, the light along the first light path L3_1 maintains the property of being polarized in the first direction and thus may be not transmitted through the first polarizer 612. The light along the second light path L3_2 may lose the property of being polarized in the first direction, and thus may be partially transmitted through the first polarizer 612 and absorbed by the light-receiving element 611. Accordingly, most of the first absorbed light AL3 may be the light along the second light path L3_2. In the first period PERIOD1, the PPG sensor 520 may generate the first PPG signal PPGS1 corresponding to an amount of the light (e.g., the first absorbed light AL3) absorbed by the light-receiving element 611. As described above, the amount of light absorbed may change in real-time according to the pulse of the user.

Referring to FIG. 9B, in the second period PERIOD2, the light-receiving module 610 may receive light along a third light path L3_3 and a fourth light path L3_4. The light-receiving module 610 may absorb light through the first polarizer 612 polarized in the first direction different from the second direction. Accordingly, as described above, the light along the third light path L3_3 maintains the property of being polarized in the first direction and thus may be not transmitted through the first polarizer 612. The light along the fourth light path L3_4 may lose the property of being polarized in the first direction, and thus, may be partially transmitted through the first polarizer 612 and absorbed by the light-receiving element 611. Accordingly, most of the second absorbed light AL4 may include the light along the fourth light path L3_4. In the second period PERIOD2, the PPG sensor 520 may generate the second PPG signal PPGS2 corresponding to an amount of the light (e.g., the second absorbed light AL4) absorbed by the light-receiving element 611. As described above, the amount of light absorbed may change in real-time according to the pulse of the user.

The first PPG signal PPGS1 and the second PPG signal PPGS2 described above with reference to FIGS. 9A and 9B may be different from each other due to the difference between the driving currents. The electronic device according to the inventive concepts may be configured to distinguish the skin tone of the user, based on the difference between two PPG signals different from each other due to the difference between the driving currents.

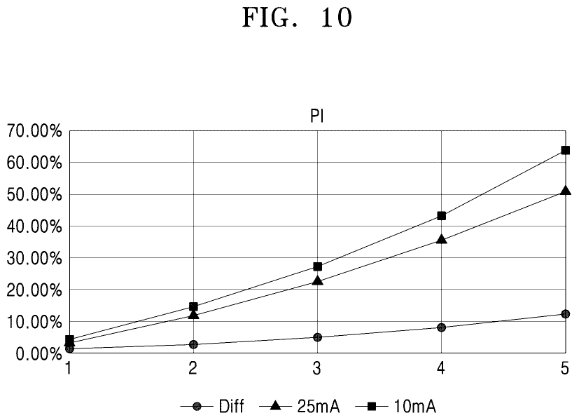

FIG. 10 is a graph showing changes in the performance of PPG according to at least one embodiment.

FIG. 10 will be described later based on the descriptions given above with reference to FIGS. 9A and 9B.

FIG. 10 is a graph showing a difference Diff between the PI of the first PPG signal PPGS1 generated based on the first driving current DS1 (10 mA) and the PI of the second PPG signal PPGS2 generated based on the second driving current DS2 (25 mA). A horizontal axis of the graph indicates the skin tone of the user. Referring to FIG. 10, the difference Diff in PIs may increase as the skin tone of the user becomes darker. The electronic device 100 (see FIG. 1) according to the inventive concepts may be configured to distinguish the skin tone of the user based on the difference between PIs of two PPG signals (e.g., the first PPG signal PPGS1 and the second PPG signal PPGS2) generated based on the driving currents (e.g., the first driving current DS1 and the second driving current DS2) different from each other.

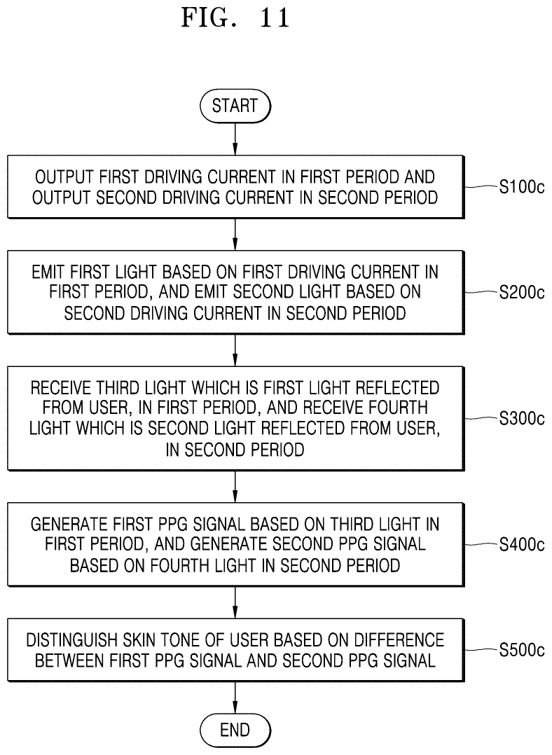

FIG. 11 is a flowchart for describing the operating method of the electronic device, according to at least one embodiment.

FIG. 11 is a flowchart for describing the operating method of the electronic device described above with reference to FIGS. 9A and 9B. Accordingly, FIG. 11 may be understood with reference to FIGS. 9A and 9B.

Referring to FIG. 11, in S100c, the electronic device outputs the first driving current in the first period and output the second driving current in the second period. The first period and the second period are different time periods, and the second period may be performed after the first period, but the inventive concepts are not limited thereto. For example, the first period may be performed after the performance of the second period. That is, the terms “first period” and “second period” are only used to distinguish in time periods in which two PPG signals are respectively generated with different driving currents based on a light-receiving module and a light-emitting module, and the inventive concepts is not limited to orders of the first period and the second period.

In S200c, the electronic device emit first lights based on a first driving current in the first period, and emits second light based on a second driving current in the second period.

In S300c, the electronic device receives, in the first period, third light that is the first light reflected from the user, and receives, in the second period, fourth light that is the second light reflected from the user.

In S400c, the electronic device generates the first PPG signal based on the third light in the first period, and may generate the second PPG signal based on the fourth light in the second period.

In S500c, the electronic device distinguishes the skin tone of the user, based on the difference between the first PPG signal and the second PPG signal.

As described above, the electronic device according to the inventive concepts may distinguish the skin tone of the user, based on a difference between two PPG signals. For convenience of explanation, it is described that the skin tone of the user may be distinguished by using the electronic device, but the inventive concepts is not limited thereto. For example, by using the method described above, skin tones of other people, as well as the user of the electronic device, may be distinguished. The electronic device according to the inventive concepts may improve the PPG performance by distinguishing the skin tone of the user and determining an ultraviolet absorption rate according to the skin tone, and may also determine the ultraviolet absorption rate according to the skin tone, calculate a degree of ultraviolet-dependent vitamin synthesis, and provide relevant information to the user.

As described above, example embodiments have been disclosed in the drawings and the specification. Although embodiments have been described herein using specific terms, such terms have been used only to describe the inventive concepts and are not intended to limit meanings or the scope of the inventive concepts written in the following claims. Therefore, those skilled in the art would understand that various embodiments and other equivalent embodiments may be made based on the inventive concepts. Accordingly, the technical scope of the inventive concepts will be determined according to the following claims.

While the inventive concepts have been particularly shown and described with reference to embodiments thereof, it will be understood that various changes in form and details may be made therein without departing from the spirit and scope of the following claims.

What is claimed is:

1. An electronic device comprising:
a light-emitter comprising a first polarizer polarized in a first direction, the light-emitter configured to emit first light polarized in the first direction through the first polarizer to a skin of a user;
a first light-receiver comprising a second polarizer polarized in a second direction different from the first direction, the first light-receiver configured to receive second light reflected from the skin of the user through the second polarizer;
a second light-receiver configured to receive third light reflected from the skin of the user; and
processing circuitry configured to generate a first photoplethysmogram (PPG) signal based on the second light received by the first light-receiver, to generate a second PPG signal based on the third light received by the second light-receiver, and to distinguish a skin tone of the user based on a difference between the first PPG signal and the second PPG signal.

2. The electronic device of claim 1, wherein the second light-receiver comprises a third polarizer polarized in the first direction, and is further configured to receive the third light through the third polarizer.

3. The electronic device of claim 1, wherein the first direction is perpendicular to the second direction.

4. The electronic device of claim 1, wherein the processing circuitry is configured to distinguish the skin tone of the user based on a difference between a first perfusion index (PI) and a second PI,
the first PI comprises a ratio of an alternating current component to a direct current component included in the first PPG signal, and
the second PI comprises a ratio of an alternating current component to a direct current component included in the second PPG signal.

5. The electronic device of claim 4, wherein the processing circuitry is configured to distinguish the skin tone of the user as a darker skin tone as the first PI increases with respect to the second PI.

6. The electronic device of claim 1, wherein the processing circuitry is configured to distinguish the skin tone of the user based on a difference between a second signal-to-noise ratio (SNR) of an alternating current component included in the second PPG signal and a first SNR of an alternating current component included in the first PPG signal.

7. An electronic device comprising:
a first light-emitter comprising a first polarizer polarized in a first direction, the light-emitter configured to emit first light polarized in the first direction through the first polarizer to a skin of a user;
a second light-emitter configured to emit second light to the skin of the user;
a light-receiver comprising a second polarizer polarized in a second direction different from the first direction, the light-receiver configured to receive third light, which is the first light reflected from the user, through the second polarizer, and to receive fourth light through the second polarizer, the third light including the first light reflected from the skin of the user and the fourth light including the second light reflected from the skin of the user; and
processing circuitry configured to generate a first photoplethysmogram (PPG) signal based on the third light received by the light-receiver, to generate a second PPG signal based on the fourth light received by the light-receiver, and to distinguish a skin tone of the user based on a difference between the first PPG signal and the second PPG signal.

8. The electronic device of claim 7, wherein
the second light-emitter comprises a third polarizer polarized in the second direction, and is further configured to emit the second light through the third polarizer such that the second light is polarized in the second direction.

9. The electronic device of claim 7, wherein the first direction is perpendicular to the second direction.

10. The electronic device of claim 7, wherein the electronic device is configured such that
in a first period, the first light-emitter emits the first light, the light-receiver receives the third light, and the processing circuitry generates the first PPG signal based on the third light, and
in a second period different from the first period, the second light-emitter emits the second light, the light-receiver receives the fourth light, and the processing circuitry generates the second PPG signal based on the fourth light.

11. The electronic device of claim 7, wherein the processing circuitry is further configured to:
output a first driving current to the first light-emitter to enable the first light and output a second driving current to the second light-emitter to enable the second light, and
wherein an intensity of the first driving current and an intensity of the second driving current are identical to each other.

12. The electronic device of claim 7, wherein the processing circuitry is configured to distinguish the skin tone of the user based on a difference between a first perfusion index (PI) and a second PI,
the first PI comprises a ratio of an alternating current component to a direct current component included in the first PPG signal, and
the second PI comprises a ratio of an alternating current component to a direct current component included in the second PPG signal.

13. The electronic device of claim 12, wherein the processing circuitry is configured to distinguish the skin tone of the user as a darker skin tone as the first PI increases with respect to the second PI.

14. The electronic device of claim 7, wherein
the processing circuitry is configured to distinguish the skin one of the user based on a difference between a second signal-to-noise ratio (SNR) of an alternating current component included in the second PPG signal and a first SNR of an alternating current component included in the first PPG signal.

15. An electronic device comprising:
a light-emitter configured to emit first light to a skin of a user based on a first driving current during a first period, and to emit second light to the skin of a user based on a second driving current during a second period different from the first period;
a light-receiver configured to receive third light in the first period and to receive fourth light in the second period, the third light including the first light reflected from the skin of the user, and the fourth light including the second light reflected from the skin of the user; and
processing circuitry configured to output the first driving current in the first period, to output the second driving current in the second period, to generate a first photoplethysmogram (PPG) signal based on the third light received by the light-receiver in the first period, to generate a second PPG signal based on the fourth light received by the light-receiver in the second period, and to distinguish a skin tone of the user based on a difference between the first PPG signal and the second PPG signal,
wherein the second driving current has an intensity different from an intensity of the first driving current.

16. The electronic device of claim 15, wherein the processing circuitry is configured to distinguish the skin tone of the user based on a difference between a first perfusion index (PI) and a second PI,
the first PI comprises a ratio of an alternating current component to a direct current component included in the first PPG signal, and
the second PI comprises a ratio of an alternating current component to a direct current component included in the second PPG signal.

17. The electronic device of claim 15, wherein
the light-emitter comprises a first polarizer polarized in the first direction such that the first light and the second light are polarized in the first direction, and
the light-receiver is configured to receive the third light and the fourth light through a second polarizer polarized in a second direction different from the first direction such that the third light and the fourth light are polarized in the second direction.

18. The electronic device of claim 17, wherein the first direction is perpendicular to the second direction.

19. The electronic device of claim 17, wherein
the processing circuitry is configured to distinguish the skin tone of the user based on a difference between a first perfusion index (PI) and a second PI,
the first PI comprises a ratio of an alternating current component to a direct current component included in the first PPG signal, and
the second PI comprises a ratio of an alternating current component to a direct current component included in the second PPG signal.

20. The electronic device of claim 15, wherein
the processing circuitry is configured to distinguish the skin tone of the user based on a difference between a second signal-to-noise ratio (SNR) of an alternating current component included in the second PPG signal and a first SNR of the alternating current component included in the first PPG signal.
